(19) United States
(12) Patent Application Publication
(10) Pub. No.: US 2007/0213642 A1
(43) Pub. Date: Sep. 13, 2007

(54) Device and method for measuring parameters of intradiscal space

(75) Inventor: Tom J. Francis, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI Holdings, Inc.

(21) Appl. No.: 11/352,257

(22) Filed: Feb. 13, 2006

(51) Int. Cl. A61B 5/103

(52) U.S. Cl. 600/594

(57) ABSTRACT

An intradisc sizer for measuring dimensions in an intradiscal space is provided. The intradisc sizer may comprise a longitudinal element having a longitudinal axis, proximate and distal ends, and an axially concentric bore. A measuring mechanism may be disposed in a compressed state in the axially concentric bore of the longitudinal element. An actuator mechanism may be positioned at the proximate end of the longitudinal element, and the actuator mechanism may be capable of causing the measuring mechanism to protrude out of the distal end of the axially concentric bore and attain an expanded state. Also, methods to measure at least one dimension in an intradiscal space using the intradisc sizers are provided.

### FIELD OF THE INVENTION

Embodiments of the invention relate to devices for measuring dimensions of an intradiscal space. More particularly, embodiments of the invention relate to devices having expandable and compressible measuring mechanisms for measuring dimensions of an intradiscal space.

### BACKGROUND OF THE INVENTION

The intervertebral disc functions to stabilize the spine and to distribute forces between vertebral bodies. The intervertebral disc primarily includes three structures: the nucleus pulposus; the annulus fibrosis; and two vertebral end-plates. The nucleus pulposus is an amorphous hydrogel in the center of the intervertebral disc. The annulus fibrosis, which is composed of highly structured collagen fibers, maintains the nucleus pulposus within the center of the intervertebral disc. The vertebral end-plates, composed of hyalin cartilage, separate the disc from adjacent vertebral bodies and act as a transition zone between the hard vertebral bodies and the soft disc.

Intervertebral discs may be displaced or damaged due to trauma, disease, and the normal aging process. One way to treat a displaced or damaged intervertebral disc is by surgical removal of a portion or all of the intervertebral disc, including the nucleus and the annulus fibrosis. However, the removal of the damaged or unhealthy disc may allow the disc space to collapse, which may lead to instability of the spine, abnormal joint mechanics, nerve damage, and severe pain. Therefore, after removal of the disc, a spinal implant such as a prosthetic nucleus, artificial disc (i.e. total disc replacement), or fusion cage may be implanted in order to replace the removed nucleus or annulus, or a portion thereof.

Because the spinal implant is replacing all or a portion of the intervertebral disc, it may be desirable to size the spinal implant according to the natural dimensions and geometry of the intervertebral disc that is to be replaced or augmented.

The description herein of problems and disadvantages of known devices and methods is not intended to limit the invention to the exclusion of these known entities. Indeed, embodiments of the invention may include one or more of the known devices and methods without suffering from the disadvantages and problems noted herein.

### SUMMARY OF THE INVENTION

What is needed are devices and methods for measuring dimensions of an intradiscal space. What also is needed are devices and methods for measuring dimensions of an intradiscal space in a minimally invasive manner. Additionally, what is needed are devices and methods for measuring dimensions of an intradiscal space that are relatively simple and fast to use. Furthermore, devices are needed for determining if a given spinal implant is appropriate for use in an intradiscal space. Embodiments of the invention solve some or all of these needs, as well as additional needs.

Therefore, in accordance with the embodiments, there is provided an intradisc sizer. The intradisc sizer may comprise a longitudinal element having a longitudinal axis, proximate and distal ends, and an axially concentric bore. A measuring mechanism may be disposed in a compressed state in the axially concentric bore of the longitudinal element. An actuator mechanism may be positioned at the proximate end of the longitudinal element, and the actuator mechanism may be capable of causing the measuring mechanism to protrude out of the distal end of the axially concentric bore and attain an expanded state.

Embodiments also provide a method of measuring at least one dimension of an intradiscal space. An intradisc sizer is provided according to embodiments described herein. The intradiscal space may be accessed and the distal end of the intradisc sizer may be advanced to a position proximate to the intradiscal space. The actuator mechanism of the sizer may be activated so that the measuring mechanism protrudes out of the distal end of the axially concentric bore and attains an expanded state in the intradiscal space. While the measuring mechanism is in an expanded state, the intradiscal space may be imaged in order to determine if the measuring mechanism has expanded fully.

These and other features and advantages of the present invention will be apparent from the description provide herein.

### BRIEF DESCRIPTION OF THE DRAWINGS

FIG. 1 is an illustration of an exemplary intradisc sizer in accordance with the embodiments.

FIG. 2 is an illustration of an exemplary intradisc sizer in accordance with the embodiments.

FIG. 3 is an illustration of an exemplary method of using an intradisc sizer in accordance with the embodiments.

### DETAILED DESCRIPTION OF THE EMBODIMENTS

The following description is intended to convey a thorough understanding of the various embodiments of the invention by providing a number of specific embodiments and details involving intradisc sizers. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the phrase “intradiscal space” refers to any volume or void between two adjacent vertebrae. The intradiscal space may be the volume inside of the annulus fibrosis of the intervertebral disc. Alternatively, the intradiscal space also may include the annulus fibrosis itself. The intradiscal space also may comprise all or only a portion of the volume between the adjacent vertebrae.

The phrase “intradisc sizer” refers to an instrument or device for measuring at least one dimension of an intradiscal space. Embodiments herein describe exemplary intradisc sizers that may be used, for example, to measure the height, posterior-anterior width, and lateral width of an intradiscal space.

The phrase “measuring mechanism” refers to a compressible and expandable device for measuring at least one dimension. Measuring mechanisms do not include intervertebral disc devices such as nucleus replacement devices, prosthetic nucleus implants, artificial disc (i.e. total disc replacement) devices, fusion cages, annulus replacements, annulus patches or grafts, and so forth.

It is a feature of an embodiment of the present invention to provide an intradisc sizer. The intradisc sizer may comprise a longitudinal element having a longitudinal axis, proximate and distal ends, and an axially concentric bore. A measuring mechanism may be disposed in a compressed state in the axially concentric bore of the longitudinal element. An actuator mechanism may be positioned at the proximate end of the longitudinal element, and the actuator mechanism may be capable of causing the measuring mechanism to protrude out of the distal end of the axially concentric bore and attain an expanded state. Preferably, the measuring mechanism is capable of measuring a dimension within the range of from about 1 millimeter to about 35 millimeters, more preferably, within the range of from about 5 millimeters to about 30 millimeters, in order to be useful for measuring the dimensions of a common intradiscal space.

The longitudinal element may have a longitudinal axis, proximate and distal ends, and an axially concentric bore. Therefore, the longitudinal element may be any applicable cannula, catheter, trocar, or other hollow or tubular structure, in accordance with the guidelines provided herein. Generally, the axially concentric bore inside of the longitudinal element and the longitudinal element may have any applicable cross-section, for example a circle, square, rectangle, ellipse, and so forth. Preferably, the longitudinal element is dimensioned such that it is able to be easily advanced to a position proximate to an intradiscal space. More preferably, the distal end of the longitudinal element is dimensioned such that it is capable of being inserted within the intradiscal space.

For example, it may be preferred that the longitudinal element have a height of no more than about 5 millimeters in its minor cross-sectional axis. This may be advantageous in order to ensure that the longitudinal element can be inserted into the confines of the intradiscal space. More preferably, the height is no more than about 4 millimeters in its minor cross-sectional axis. Most preferably, the height is no more than about 3 millimeters. The term “height” denotes the shortest (if more than one) of the primary dimensions of the cross-sectional geometry of the longitudinal element. For example, for a longitudinal element that is circular in cross-section, the diameter is the height; for a square, the length of one side of the square is the height; for a rectangle, the length of the shorter set of sides is the height; for an ellipse, the length of the minor axis is the height; and so forth.

It also may be preferred that the longitudinal element have a length within the range of from about 10 centimeters to about 20 centimeters. More preferably, the length is from about 12 centimeters to about 14 centimeters. Most preferably, the length is about 13 centimeters. In another embodiment, the entire length of the intradisc sizer, including the longitudinal element, is about 20 centimeters. Sizing the longitudinal element within these dimension may facilitate insertion of the longitudinal element into an intradiscal space by ensuring that the longitudinal element is long enough to reach from the point where it is inserted into the body to the intradiscal space. For example, if the intradisc sizer approaches the intradiscal space from a posterior approach, a relatively short longitudinal element may be sufficient. However, if the intradisc sizer approaches the intradiscal space from a trans-foraminal or anterior approach, a relatively long longitudinal element may be more appropriate. Also, the longitudinal element of the intradisc sizer preferably will not be excessively long as this may make the instrument unwieldy and difficult to use. Alternatively, the longitudinal element may be similar to a sheath and cover only the measuring mechanism, and may itself be positionable and inserted through a second longitudinal element. One skilled in the art will be able to choose an appropriate length for the longitudinal element, in accordance with the guidelines provided herein.

Additionally, the longitudinal element may be manufactured using any appropriate material. For example, medical plastics such polyvinyl chlorides, polypropylenes, polystyrenes, acetal copolymers, polyphenyl sulfones, polycarbonates, acrylics, silicone polymers, and mixtures and combinations thereof may be used to manufacture the longitudinal element. Metals and metal alloys such as titanium, titanium alloys, tantalum, tantalum alloys, stainless steel alloys, cobalt-based alloys, cobalt-chromium alloys, cobalt-chromium-molybdenum alloys, niobium alloys, and zirconium alloys also may be used to fabricate the longitudinal element. One who is skilled in the art will be able to choose an appropriate material for fabricating the longitudinal element based on the guidelines provided herein.

The measuring mechanism may be any appropriate mechanism for measuring at least one dimension of an intradiscal space, in accordance with the guidelines provided herein. The measuring mechanism may be disposed in the axially concentric bore of the longitudinal element in a compressed state. Upon activation, the measuring mechanism may protrude out of the distal end of the longitudinal element and expand. Preferably, deactivating the actuator mechanism may cause the measuring mechanism to recede into the axially concentric bore. In doing so, the measuring mechanism may revert or contract to a compressed state. Preferably, the measuring mechanism has a substantially reduced cross-section in the compressed state, compared to its expanded state. Therefore, the measuring member preferably may be in a compressed state to facilitate accessing the intradiscal space and inserting the measuring mechanism into the confines of the intradiscal space.

In order to measure at least one dimension of the intradiscal space, before inserting the measuring mechanism into the body, the maximum cross-section or dimension to which the measuring mechanism is able to expand preferably is measured. After insertion of the measuring mechanism into the intradiscal space and expansion of the measuring mechanism, an imaging technique such as, but not limited to, fluoroscopy (e.g. X-ray, CT scan, C-arm fluoroscopy, etc.) may be used to examine the extent to which the measuring mechanism has expanded inside of the intradiscal space. If the measuring mechanism has expanded fully, then it can be inferred that the dimension being measured is at least as large as the pre-determined cross-section or dimension of the fully expanded measuring mechanism. If the measuring mechanism has not expanded fully because it is constrained by the surfaces of the intradiscal space (e.g., the vertebral endplates), then it can be inferred that the dimension being measured is smaller or shorter that the pre-determined cross-section or dimension of the fully expanded measuring mechanism.

Because the measuring mechanism may take a number of different forms and may be oriented in a number of different directions in the intradiscal space, the measuring mechanism may be capable of measuring many different dimensions or parameters of an intradiscal space. For example, a measuring mechanism that is able to expand in only one dimension may be oriented so that it is capable of expanding in a direction parallel to the spinal column in order to measure the intradiscal height, or may be oriented so that it is capable of expanding in a direction perpendicular to the spinal column in order to measure a dimension of the intradiscal space's cross-section, such as its posterior-anterior width or lateral width.

In one embodiment of the invention, the measuring mechanism is a coil spring. A compressed state of the coil spring means that the coil spring is wound up to a diameter substantially less than the coil spring's diameter in an unwound, or resting, state. An expanded state of the coil spring corresponds to the coil spring in an unwound, or resting, state. Preferably, the coil spring is capable of expanding to a diameter within the range of from about 1 millimeter to about 35 millimeters.

The coil spring may be oriented relative to the longitudinal element so that it winds, for example, in either a plane substantially parallel to the longitudinal axis of the device or a plane substantially perpendicular to the longitudinal axis. Additionally, by orienting the longitudinal axis (e.g. rotating the longitudinal element) the coil spring can be brought into various different alignments or orientations with respect to the intradiscal space and spinal column. For example, if the coil spring winds in a plane substantially parallel to the device's longitudinal axis, the coil spring may be oriented, for example, so that the spring winds in a plane parallel to the spinal column. In this orientation, the measuring mechanism may be used to determine the height of the intradiscal space. Alternatively, the coil spring may be oriented, for example, so that the spring winds in a plane perpendicular to the spinal column. In this orientation, the measuring mechanism may be used to determine the width, for example posterior-anterior or lateral width, of the intradiscal space.

In another embodiment of the invention, the measuring mechanism is a flexible ring. In a compressed state, the flexible ring may be in an elliptical shape having a major and a minor axis. In an expanded shape, the flexible ring may be in a substantially more circular shape (i.e. the major and minor axis are more nearly equal to each other). To transform from an elliptical compressed state to a substantially more circular expanded state, the ring's minor axis preferably increases or expands in length. Therefore, it is the minor axis of the flexible ring that preferably may be used to measure a dimension of the intradiscal space. Also, the flexible ring preferably flexes in a plane substantially parallel to the longitudinal axis of the longitudinal element. In a preferred embodiment, the ring's minor axis is capable of expanding to a length in the range of from about 1 millimeter to about 35 millimeters, more preferably within the range of from about 5 millimeters to about 30 millimeters.

The measuring mechanism that is a flexible ring may be brought into various different alignments or orientations with respect to the intradiscal space, for example, by rotating the longitudinal element of the intradisc sizer. The flexible ring may be oriented, for example, so that the ring expands in a plane parallel to the spinal column. In this orientation, the measuring mechanism may be used to determine the height of the intradiscal space. Alternatively, the flexible ring may be oriented so that the ring expands in a plane perpendicular to the spinal column. In this orientation, the measuring mechanism may be used to determine the width, either posterior-anterior or lateral, of the intradiscal space.

The flexible ring and coil spring each may be made of any appropriate strip or piece of flexible material. Metallic alloys are a preferred material for fabrication of the flexible ring and coil spring, such as stainless steels and other medical alloys. In another embodiment, shape memory alloys are the preferred material for fabrication of the flexible ring and coil spring. Shape memory alloys include, but are not limited to, alloys of nickel titanium (“nitinol”), copper-zinc-aluminum, copper-aluminum-nickel, iron-manganese-silicon, silver-cadmium, gold-cadmium, copper-tin, copper-zinc, copper-zinc-silicon, copper-zinc-tin, indium-titanium, nickel-aluminum, iron-platinum, manganese-copper, and iron-manganese-silicon. In another preferred embodiment, the flexible ring and coil spring member may be fabricated of an appropriate polymeric material, such as a silicone-based polymer or a polyurethane (e.g. the silicone-urethane copolymer PurSil®, commercially available from The Polymer Technology Group, Berkeley, Calif.). In the case of a polymeric material, it may be desirable to include one or more radiographic markers in the measuring mechanism in order to increase its visibility in radiographic images. Thus, radiographic markers such as tantalum beads, strips, and so forth may be embedded in the polymeric materials. The actuator may be any appropriate mechanical, electrical, hydraulic, or other device that is capable of causing the measuring mechanism to protrude out of the distal end of the longitudinal element and attain an expanded state. For example, a mechanical or hydraulic actuator may force the measuring mechanism out of the distal end of the longitudinal element. Upon exiting the longitudinal element, the measuring mechanism preferably automatically attains an expanded state because it is no longer constrained by the concentric bore of the longitudinal element. The actuator may take any of a number of different configurations. One who is skilled in the art will recognize various configurations which the actuator may take, in accordance with the guidelines provided herein.

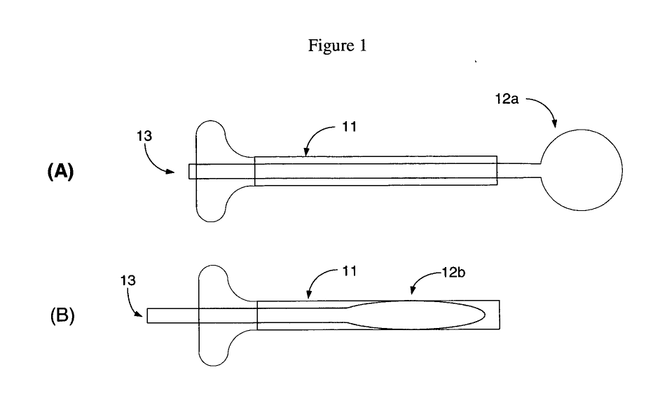

For example, the actuator may be a simple push-rod, as is illustrated in FIG. 1, embodiments A and B. A longitudinal element 11 is provided. In embodiment A, the measuring mechanism comprising a flexible ring 12a has exited the distal end of the longitudinal element and is in an expanded state. As can be seen, the expanded state may correspond to a more nearly circular state. In embodiment B, the flexible ring 12b has been retracted into the longitudinal element and is in a compressed state. As can be seen, the compressed state may correspond to a more nearly elliptical or oval shape. An actuator mechanism comprising a push-rod 13 is provided at the proximate end of the longitudinal element. The push-rod 13 may be attached to the flexible ring 12a and 12b. To activate the intradisc sizer, the push-rod may be pushed towards the distal end of the longitudinal element, thereby causing the flexible ring 12a to exit out of the distal end of the longitudinal element 11 and allowing the flexible ring 12a to attain an expanded state (embodiment A). To deactivate the intradisc sizer, the push-rod may be retracted, thereby causing the flexible ring 12b to retract into the longitudinal element 11 and attain a compressed state (embodiment B). Any actuating mechanism may be used that is capable of moving the flexible ring from a retracted state 12b to an expanded state 12a.

In an alternative embodiment, the intradisc sizer may be used to determine if a particular spinal implant is appropriate for use in an intradiscal space. In this embodiment, the flexible ring 12a in its expanded state is in a shape approximating that of a cross-section of the spinal implant. For example, if the spinal implant is a cylindrical fusion cage, then the flexible ring in its expanded state may be circular. If the spinal implant is a kidney-shaped nucleus or total disc replacement device (e.g., the Nautilus® series of disc devices produced by Medtronic, Minneapolis, Minn.), then the flexible ring in its expanded state also may have a kidney-like shape. If the flexible ring is capable of expanding fully or at least substantially so, then it may be concluded that the spinal implant is not too large for the intradiscal space and is appropriate for use therein.

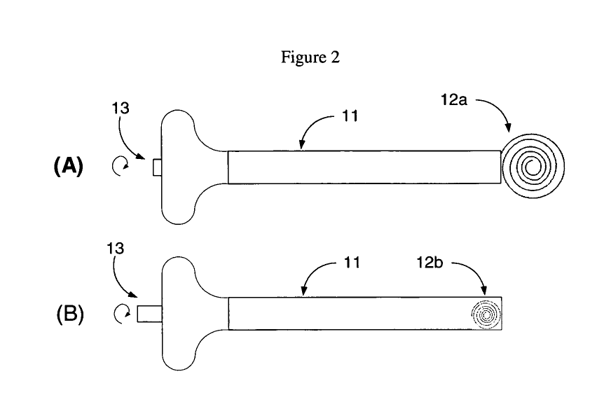

In FIG. 2, embodiments A and B, a longitudinal element 11 is provided. In embodiment A, the measuring mechanism comprising a coil spring 12a has existed the distal end of the longitudinal element and has unwound to an expanded state. In embodiment B, the coil spring 12b has been rewound to a compressed state and retracted into the longitudinal element. An actuator mechanism comprising a rotatable dial 13 is provided at the proximate end of the longitudinal element. The rotatable dial 13 may be attached to the coil spring 12a and 12b. To activate the intradisc sizer, the rotatable dial may be rotated to cause the coil spring to unwind, and the rotatable dial may push the coil spring distally out of the end of the longitudinal element 11 (embodiment A). To deactivate the intradisc sizer, the rotatable dial may be rotated again, thereby causing the flexible ring 12b to rewind to a compressed state and retract into the longitudinal element 11 (embodiment B). Any actuating mechanism may be used that is capable of moving the coil spring from a retracted state 12b to an expanded state 12a.

Various parameters of the intradiscal space may be determined by use of the devices described herein. Preferably, the height of the intradiscal space may be measured. Measuring the height of the intradiscal space may include measuring the anterior, middle, and posterior height of the disc space, as these three measurements may differ, even in the same intradiscal space. Additionally, the wedge angle of the intradiscal space may be calculated from the anterior, middle, and posterior height of the disc space. Another parameter that may be measured is the width, both posterior-anterior and lateral, of the intradiscal space. Determination of parameters of the disc space may enable customization and sizing of a spinal implant to an intradiscal space.

In order to measure the posterior-anterior and lateral width of the intradiscal space, the measuring mechanism preferably is capable of expanding to a sufficiently large expanded state. For example, for an intradiscal space in the cervical portion of the spine, the posterior-anterior width of the measuring mechanism in its expanded state preferably is from about 5 millimeters to about 7 millimeters, and the lateral width of the measuring mechanism in its expanded state preferably is from about 6 millimeters to about 8 millimeters. For an intradiscal space in the lumbar portion of the spine, the posterior-anterior width of the measuring mechanism in its expanded state preferably is from about 15 millimeters to about 30 millimeters, and the lateral width of the measuring mechanism in its expanded state preferably is from about 20 millimeters to about 35 millimeters. Skilled artisans are capable of designing a suitable measuring mechanism, depending on the particular area of the spine being measured, using the guidelines provided herein.

The systems and methods described herein may be advantageously used to determine the height, posterior-anterior width, lateral width, and other parameters of an intradiscal space prior to implantation of a spinal implant to replace all or part of the nucleus and annulus of the intervertebral disc. The spinal implant may be, for example, a fusion cage, artificial disc, or prosthetic disc nucleus. A snug fit between the spinal implant and the intradiscal space is thought to be desirable because of the reduced possibility of implant rotation, reduced possibility of excessive implant movement inside the disc space, increased contact between the vertebral end plates and implant, and increased annulus tension. Therefore, a correctly sized spinal implant may be more likely to achieve a desirable clinical result than would be an incorrectly sized implant.

In another embodiment, excess tissue may be removed before implantation of the spinal implant. Because spinal implants often are manufactured pre-surgery, it may be necessary to shape the intradiscal space to fit the implant that it is be implanted. Measuring parameters of the intradiscal space therefore may enable a surgeon to determine what, if any, excess tissue should be removed prior to implantation of the spinal implant. This may lead to a closer correlation in size between the intradiscal space and the spinal implant, and a more desirable clinical outcome.

An intradisc sizer as has been described may be used by first accessing the intradisc space. Preferably, this may be done in a minimally invasive fashion in order to avoid perturbing adjacent tissues and in order to reduce a patient's recovery time. Then, the distal end of the intradisc sizer may be advanced to a position proximate to the intradiscal space. Alternatively, the distal end of the intradisc sizer may be inserted slightly into the intradiscal space. The actuator mechanism may be activated in order to cause the measuring mechanism of the device to protrude out of the distal end of the longitudinal element and attain an expanded state in the intradiscal space.

While the measuring mechanism is in an expanded state, the intradiscal space may be imaged using, for example, a fluoroscopic imaging technique. It may be advantageous to include a dimensional reference in the image in order to normalize the dimensions of the image. The relevant dimensions of the expanded measuring mechanism obtained thereby may be compared to the known dimensions of the measuring mechanism in its fully expanded state in order to determine if the intradisc sizer has been able to fully expand. The intradisc sizer then may be deactivated so that the measuring mechanism returns or contracts to a compressed state and recedes into the longitudinal element. The intradisc sizer then may be retracted from its position proximate to or inside of the intradiscal space.

If the first intradisc sizer is not able to attain a fully expanded state, then the process may be replicated with a subsequently smaller sizers until a sizer is found that can expand to its fully expanded state. Similarly, if the first intradisc sizer is able to reach its fully expanded state, then the process may be repeated with subsequently larger sizers until a sizer is found that cannot reach its fully expanded state within the confines of the intradiscal space. In either case, the approximate measurement of the relevant dimension of the intradiscal space can be determined by this trial-and-error method.

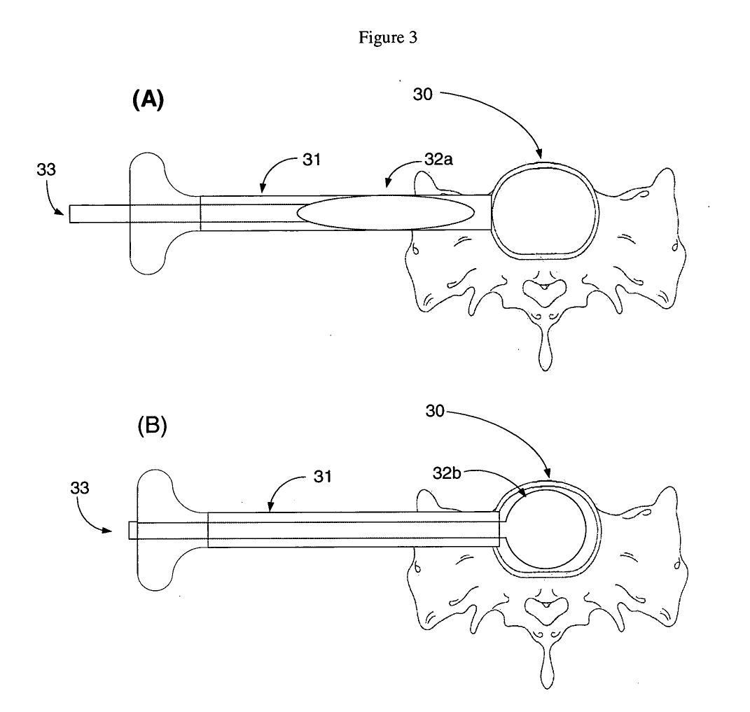

FIG. 3, embodiments A and B, illustrates an exemplary method of using an intradisc sizer. In embodiment A, an intradisc sizer comprising a longitudinal element 31, an actuator mechanism 33, and a measuring mechanism 32a is shown at a position proximate to the intradiscal space 30 after approaching the intradiscal space from a transforaminal direction. In embodiment B, the actuator mechanism 33 has been activated and has caused the measuring mechanism 32b to protrude out of the distal end of the device and attain an expanded state in the intradiscal space. In this example, the measuring mechanism is a flexible ring and has been oriented to flex in a plane perpendicular to the spinal column in order to measure a width, such as the posterior-anterior width or the lateral width, of the intradiscal space.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. An intradisc sizer, comprising:
a longitudinal element having a longitudinal axis, proximate and distal ends, and an axially concentric bore;
a measuring mechanism disposed in the axially concentric bore of the longitudinal element in a compressed state; and
an actuator mechanism positioned at the proximate end of the longitudinal element;
wherein the actuator mechanism is capable of causing the measuring mechanism to protrude out of the distal end of the axially concentric bore and attain an expanded state.

2. The device of claim 1, wherein deactivating the actuator mechanism causes the measuring mechanism to recede into the axially concentric bore at the distal end of the longitudinal element and contract to a compressed state.

3. The device of claim 1, wherein the measuring mechanism is capable of measuring a dimension within the range of from about 1 millimeter to about 35 millimeters.

4. The device of claim 1, wherein the measuring mechanism is a coil spring, and attaining an expanded state comprises the coil spring unwinding.

5. The device of claim 4, wherein the coil spring winds in a plane substantially parallel to the longitudinal axis.

6. The device of claim 4, wherein the coil spring winds in a plane substantially perpendicular to the longitudinal axis.

7. The device of claim 4, wherein the coil spring is capable of expanding to a diameter within the range of from about 1 millimeter to about 35 millimeters.

8. The device of claim 1, wherein the measuring mechanism is a flexible ring, the flexible ring in a compressed state is an elliptical shape having a major and a minor axis, and attaining an expanded state comprises the ring expanding from the elliptical shape to a substantially more circular shape.

9. The device of claim 8, wherein the flexible ring flexes in a plane substantially parallel to the longitudinal axis.

10. The device of claim 8, wherein the flexible ring's minor axis is capable of expanding to a length within the range of from about 1 millimeter to about 35 millimeters.

11. The device of claim 1, wherein the longitudinal element has a length within the range of from about 10 centimeters to about 20 centimeters.

12. A method for measuring at least one dimension of an intradiscal space, comprising:
providing an intradisc sizer as claimed in claim 1;
accessing the intradiscal space;
advancing the distal end of the intradisc sizer to a position proximate to the intradiscal space;
activating the actuator mechanism so that the measuring mechanism protrudes out of the distal end of the axially concentric bore and attains an expanded state in the intradiscal space; and
imaging the intradiscal space and the measuring mechanism in its expanded state to determine if it has expanded fully.

13. The method of claim 12, wherein the dimension is selected from the group consisting of the height, posterior-anterior width, and lateral width of the disc space.

14. The method of claim 12, wherein accessing the intradiscal space is accomplished using minimally invasive surgical techniques.

15. The method of claim 12, wherein imaging the intradisc sizer comprises a procedure selected from the group consisting of an X-ray, CT scan, and C-arm fluoroscopy.

16. The method of claim 12, further comprising:
deactivating the actuator mechanism so that the measuring mechanism recedes into the axially concentric bore and returns to a compressed state; and
retracting the intradisc sizer from its position proximate to the intradiscal space.

17. The method of claim 12, further comprising providing a plurality of devices as claimed in claim 1, wherein the plurality of devices have measuring mechanisms capable of expanding to different maximum sizes.

18. The method of claim 17, further comprising inserting the device having the measuring mechanism capable of expanding to the smallest size into the intradiscal space, and then the devices having a mechanism capable of expanding to a sequentially larger size until a device is found wherein the measuring mechanism cannot fully expand in the intradiscal space.

19. The method of claim 17, further comprising inserting the device having the measuring mechanism capable of expanding to the largest size into the intradiscal space, and then the devices having a mechanism capable of expanding to a sequentially smaller size until a device is found wherein the measuring mechanism can fully expand in the intradiscal space.
